(19) United States
(12) Patent Application Publication
(10) Pub. No.: US 2009/0076469 A1
(43) Pub. Date: Mar. 19, 2009

(54) Modular Dry Chamber Assembly

(76) Inventor: Morten Bay Alexandersen, Hornbaek (DK)

Correspondence Address: JACOBSON HOLMAN PLLC, 400 SEVENTH STREET N.W., SUITE 600, WASHINGTON, DC, 20004, US

(21) Appl. No.: 12/226,489

(22) PCT Filed: Apr. 19, 2007
(86) PCT No.: PCT/EP2007/053831

§ 371 Date: Nov. 5, 2008

Related U.S. Application Data
(60) Provisional application No. 60/793,223, filed on Apr. 20, 2006.

(30) Foreign Application Priority Data
Apr. 20, 2006 (DK) PA 2006 00541

(51) Int. Cl. A61M 1/00

(52) U.S. Cl. 604/317

(57) ABSTRACT

A dry chamber (100) for a modular drainage system, the dry chamber may comprise a downstream connector (106) which is in fluid connection with the dry chamber via a flexible annular element defining a conduit. A kit of elements comprising a dry chamber, and at least one of: a collecting bag (130); a device for draining body fluids; an annular element (140) for fluidly interconnecting the dry chamber and a device for draining fluids; and an annular element (138) for fluidly interconnecting the dry chamber and a collecting bag. Use of a flexible annular element for fluidly interconnecting a dry chamber and a bag for collecting body fluids.

### FIELD OF THE INVENTION

The present invention relates to a dry chamber for a modular drainage system. In particular the present invention relates to a dry chamber comprising a downstream connector which is in fluid connection with the dry chamber via a flexible annular element defining a conduit. Furthermore, the present invention relates to a kit of elements comprising a dry chamber, and at least one of: a collecting bag; a device for draining body fluids; an annular element for fluidly interconnecting the dry chamber and a device for draining fluids; and an annular element for fluidly interconnecting the dry chamber and a collecting bag. Moreover, the present invention relates to use of a flexible annular element for fluidly interconnecting a dry chamber and a bag for collecting body fluids.

### BACKGROUND OF THE INVENTION

Use of dry chambers in connection with draining of body fluids is known in the art. The purpose of such use is normally to prevent bacteria contained in a collecting bag from migrating from the collecting bag and to a body cavity from which the body fluid is drained.

One example may be seen in U.S. Pat. No. 4,158,362 which discloses a unidirectional valved drip chamber mounted on a flexible urinary collecting container. Further examples may be seen in DE 24 35 288, EP 0 058 829, U.S. Pat. No. 3,740,770 and EP 0 956 839.

It is an object of a preferred embodiment of the present invention to provide a dry chamber which may be replaced by a new dry chamber without changing other elements of the drainage system.

Moreover, it is an object of a preferred embodiment of the present invention to provide a dry chamber which may be maintained in a substantially upright position while a collecting bag to which the dry chamber is connected is changed from a substantially upright position to a substantially horizontal position.

### BRIEF DESCRIPTION OF THE INVENTION

In a FIRST aspect the present invention relates to a dry chamber for a modular drainage system, the dry chamber comprising:
a substantially rigid housing;
an upstream connector in fluid connection with a dripping surface from which fluid may drip inside the dry chamber when the dry chamber is positioned in a substantially upright position; and
a downstream connector fluidly connected to the dry chamber.

The dry chamber of the present invention may be inserted into a drainage system such that it is in fluid connection with a device for draining body fluids and with a collecting bag. In such situations the modularity of the dry chamber of the present invention allows the dry chamber to be replaced by a new, without replacing the device for draining body fluids and without replacing the collecting bag. As an example, this is advantageous when the dry chamber comprises an indicator which cannot be replaced without replacing the entire dry chamber.

In the context of the present invention the term “dry chamber” shall be understood as a medical device adapted to allow a fluid to drip from one surface to another, thus, providing an interruption in a flow path and preventing bacteria from migrating in an upstream direction.

In the context of the present invention the term “modular drainage system” shall be understood as a drainage system comprising two elements between which the dry chamber may be detachably connected, such that said two elements are fluidly interconnected via the dry chamber.

The housing of the dry chamber is substantially rigid to prevent a surface downstream the dripping surface from contacting the dripping surface.

The upstream connector is in fluid connection with the dripping surface such that fluid entering the upstream connector may flow along the inner surface of the fluid connection (e.g. a conduit) and towards the dripping surface when the dry chamber is positioned in a substantially upright position.

In the context of the present invention the term “substantially upright position” shall be understood as any position wherein fluid dripping from the dripping surface drips onto a downstream surface relative to the dripping surface. In the majority of the embodiments, this means that an inlet of the dry chamber is positioned above an outlet of the dry chamber.

In one embodiment the downstream connector and the dry chamber are fluidly interconnected by a flexible annular element defining a conduit and allowing the downstream connector to be elevated to a position above the dry chamber, when the dry chamber is positioned in said substantially upright position.

One advantage of the flexible annular element is that a collecting bag fluidly connected to the dry chamber via the flexible annular element may be positioned in a substantially horizontal position while the dry chamber remains in its substantially upright position. Moreover, the flexible annular element allows the collecting bag to be positioned above the dry chamber when the dry chamber is in its substantially upright position. This is useful when a patient is lifted from one bed to another, as it is common practice to place the collecting bag on the stomach or any other upper surface of the patient during the lift. In conventional systems, wherein the dry chamber and the collecting bag are integrated with or attached to each other, fluid contained in the lower part of the dry chamber may flow onto the dripping surface, whereby the latter is contaminated.

However when said flexible annular element is provided, the dry chamber may be maintained in its substantially upright position while the collecting bag is positioned on an upper surface of the patient. Accordingly, contamination of the dripping surface is avoided as fluid contained in the lower part of the dry chamber remains in said lower part of the dry chamber. It will be appreciated that the flexible annular element must be sufficiently long to allow the dry chamber to hang in a substantially upright position while the collecting bag is lying on the patient.

The flexible annular element interconnecting the dry chamber and the downstream connector may form an integral part of dry chamber.

In the context of the present invention the term “forms an integral part of” shall be understood such that if a first element—which forms an integral part of a second element—is separated from said second element, then the two elements are not adapted to be rejoint by hand and without use of tools. In some embodiments such first and second elements are monolithic i.e. fabricated as a single structure and without seams.

Alternatively, the flexible annular element interconnecting the dry chamber and the downstream connector may be detachably connected to the dry chamber. Accordingly, the dry chamber may be replaced without replacing the flexible annular element.

The upstream connector may form an integral part of the dry chamber. Moreover, the upstream connector and the dry chamber may be interconnected by a flexible annular element defining a conduit. The flexible annular element and the upstream connector may form an integral part of the dry chamber. Alternatively, the flexible annular element interconnecting the upstream connector and the dry chamber may be detachably connected to the dry chamber, thus, allowing the dry chamber to be replaced without replacing the flexible annular element.

The upstream connector may be adapted to be connected to a corresponding connector of a device for draining body fluids. Examples of such devices are indwelling catheters and external urinary sheaths.

In order to determine contamination or a specific property of the body fluids, the dry chamber may comprise an indicator for indicating a chemical or biological property of a fluid dripping from the dripping surface or in fluid present downstream the dripping surface. As an example, this may be used to determine presence of bacteria and/or blood in urine. In one embodiment the indicator is a pH-indicator which may be indicative of presence of Proteus Mirabilis as shown in WO 2006/000764 A1.

The indicator may be provided upstream and/or downstream of the dripping surface.

Moreover, the dry chamber may be adapted to allow replacement of the indicator. In order to allow replacement of the indicator, the dry chamber may be formed by an upper part and a lower part which may be detachably attached to each other. By detaching the upper and lower part, access may be provided to the indicator such that it may be replaced. In another embodiment the housing is a monolithic element which does not allow replacement of the indicator without replacing the entire dry chamber.

In order to reduce or even prevent the risk of flow of fluid from the collecting bag to the dripping surface, the dry chamber may comprise an antireflux valve for preventing fluid from flowing in an upstream direction in the dry chamber. The antireflux valve will in most embodiments be positioned downstream relative to the dripping surface, whereby the collecting bag may be elevated above the dry chamber without upstream (back)flow due to influence by gravity.

The design allowing upright position of the dry chamber while the collecting bag is positioned in a horizontal position, may be seen as a first means for preventing migration of bacteria from the collecting bag to the device for draining fluid. The antireflux valve may be seen as a second means for preventing migration of bacteria from the collecting bag to the dripping surface.

In a SECOND aspect the present invention relates to a kit of elements comprising a dry chamber having an upstream connector and a downstream connector, the kit further comprising at least one of:
a collecting bag for collecting body fluids, comprising a connector connectable to the downstream connector of the dry chamber;
a device for draining body fluids, comprising a connector adapted to be connected to the upstream connector of the dry chamber;
an annular element for fluidly interconnecting the upstream connector and a device for draining fluids; and
an annular element for fluidly interconnecting the downstream connector and a collecting bag.

Again, by providing a flexible annular element between the dry chamber and both the collecting bag and device for draining fluids, it is possible to position the collecting bag in a substantially horizontal position while maintaining the dry chamber in a substantially upright position. Thus, the collecting bag may be positioned on the stomach or any other upper surface of a lying patient while moving the patient from one bed to another, without increasing the risk of contamination of the dripping surface.

Additionally, the modularity of the kit enables one element to be replaced without replacement of other elements.

The invention according to the second aspect of the invention may comprise any feature or element of the invention according to the first aspect of the invention.

In a THIRD aspect the present invention relates to the use of a flexible annular element for fluidly interconnecting a dry chamber and a bag for collecting body fluids. Such use allows the aforementioned upright and horizontal positioning of the dry chamber and a collecting bag, respectively.

The invention according to the third aspect of the invention may comprise any feature or element of the invention according to the first aspect of the invention.

### DETAILED DESCRIPTION OF THE INVENTION

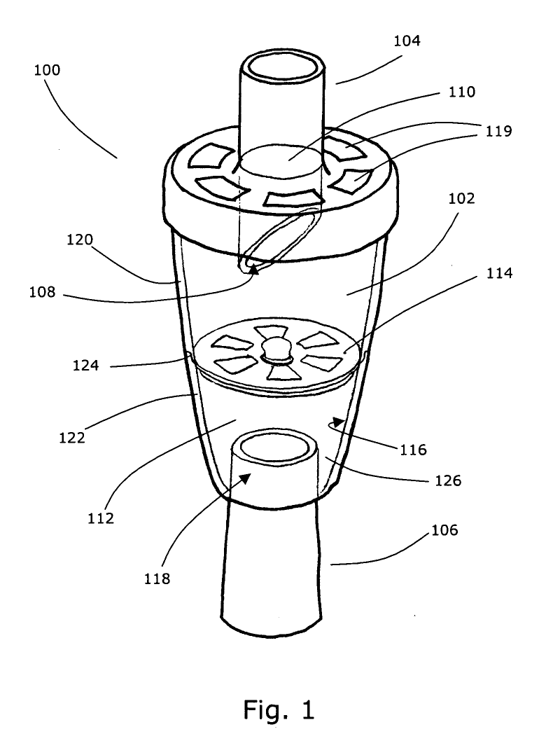

FIG. 1 discloses a dry chamber according to the invention,

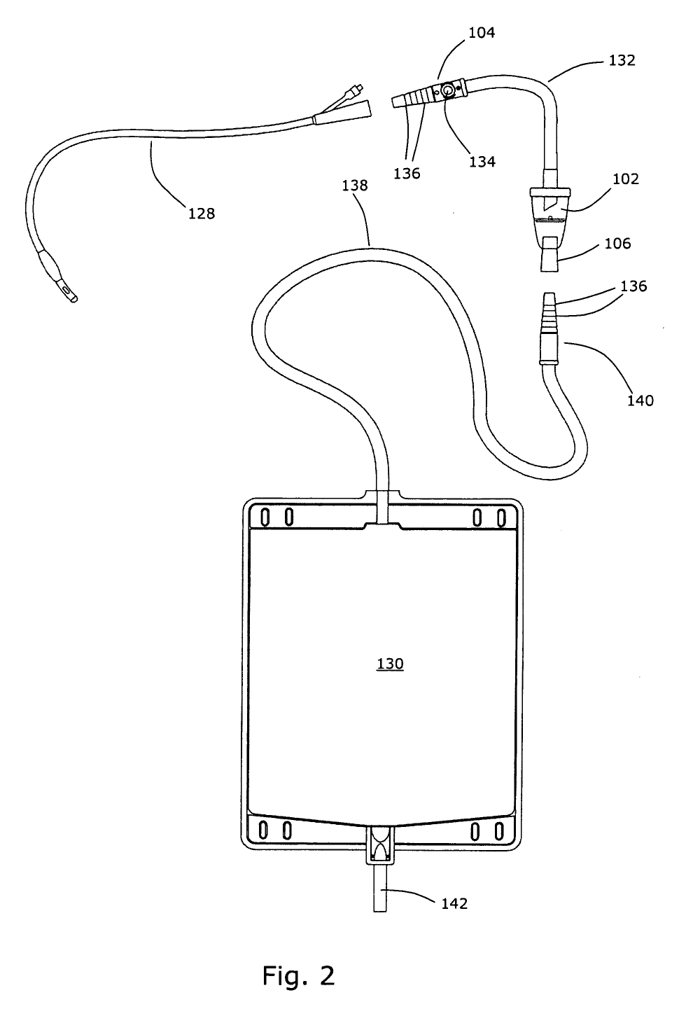

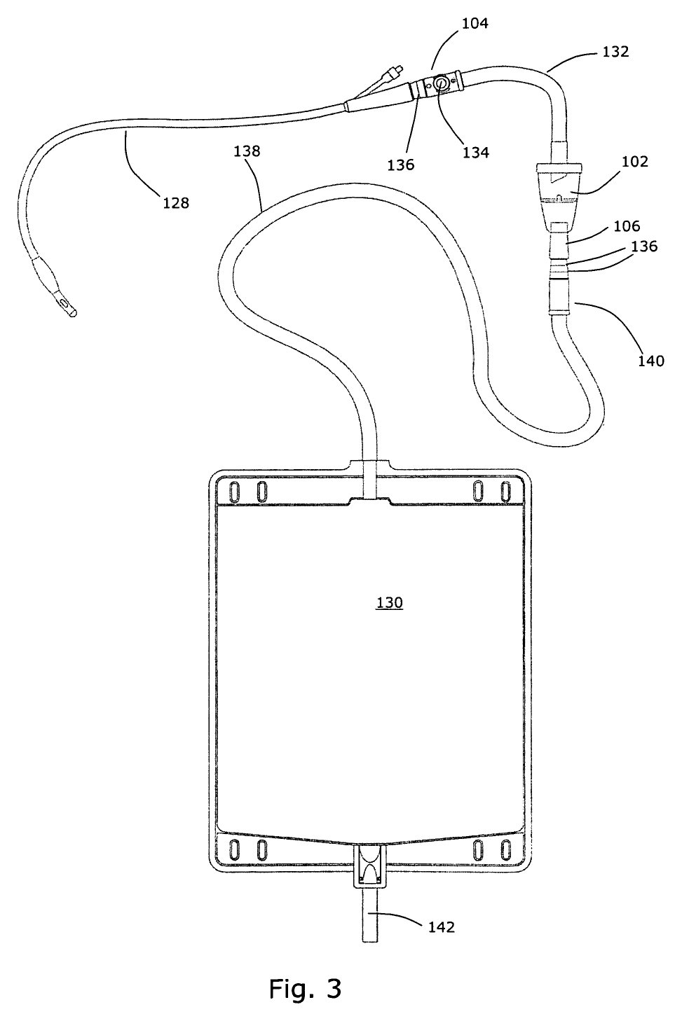

FIGS. 2 and 3 discloses a modular dry chamber system, and

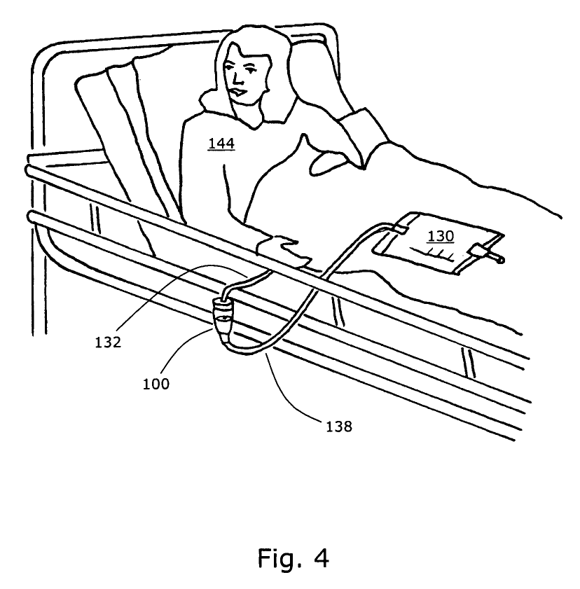

FIG. 4 discloses a use situation of the modular dry chamber system.

FIG. 1 discloses a dry chamber 100 for a modular drainage system. The dry chamber comprises a substantially rigid housing 102, an upstream connector 104 and a downstream connector 106. The dry chamber 100 comprises a dripping surface 108 which is in fluid connection with the upstream connector 104 via a conduit 110. In FIG. 1 the dry camber 100 is positioned in a substantially upright position, and, thus, fluid entering the upstream connector 104 may flow on the inner surface of the conduit 110 and drip from the dripping surface 108 towards a bottom surface 112 of the dry chamber. In the embodiment of FIG. 1, the bottom surface 112 comprises a lower part of an inner surface 116 of the housing 102 and the upper outer surface 118 of the downstream connector 106.

Furthermore, the embodiment of FIG. 1 comprises an indicator (not shown) provided in a cavity 126 defined between the inner surface 116 and the outer surface 118, i.e. downstream relative to the dripping surface 108. The indicator is adapted to indicate a chemical or biological property such as pH-level of a body fluid contacting the indicator. During use, fluid dripping from the dripping surface 108 drips onto an antireflux valve 114. The antireflux 114 is adapted to allow fluid to pass in the downstream direction, i.e. downwards in the drawing, and to prevent flow in the upstream direction, i.e. upwards in the drawing. When fluid has passed the antireflux valve 114 it continues towards the bottom surface 112, and may, thus, react with the indicator which is provided in the cavity 126.

It will be appreciated, that in other embodiments the dry chamber may advantageously be designed such that no cavity 126 is defined whereby fluid dripping onto the inner surface 116 will flow directly into the downstream connector.

The housing comprises an upper part 120 and a lower part 122 which are detachably attachable to each other. Both parts comprise abutment surfaces 124 which are adapted to provide a fluid tight seal, when the two parts are attached to each other. By detaching the two parts from each other, a user may access the indicator such that it may be replaced. Air vents 119 are provided in the upper part 120. The air vents 119 are permeable to gas and impermeable to liquid.

The upstream connector 104 is adapted to be connected to a corresponding connector of a draining device 128 for draining body fluids (see FIGS. 2 and 3). Similarly, the downstream connector 106 is adapted to be connected to a collecting bag 130 for collecting body fluids (see FIGS. 2 and 3).

In FIG. 1 a simplified version of the upstream connector 104 is disclosed compared to the upstream connector 104 of FIGS. 2 and 3. In FIGS. 2 and 3 the upstream connector 104 and the housing 102 are interconnected by a flexible upstream hose 132 (an annular element) which is permanently fixed to the housing 102 and the upstream connector 104. In other embodiments, the upstream hose is detachable attached to the housing 102 and/or the upstream connector 104. The length of the upstream hose 132 may be in the range 10-200 cm, such as in the range 50-150 cm, such as in the range 100-120 cm. The upstream connector 104 may comprise a sample port 134 through which a cannula may be inserted so as to take a sample. The outer surface of the upstream connector may comprise a plurality of corrugations 136 adapted to engage an inner surface of said corresponding connector of the draining device 128, as illustrated in FIG. 3. In FIGS. 2 and 3 the draining device comprises an indwelling catheter forming a retaining part.

The collecting bag 130 comprises a downstream hose 138 and a connector 140, which is adapted to engage the downstream connector 106 of the dry chamber 100. In the embodiment of FIGS. 2 and 3, the downstream hose 138 is permanently fixed to the collecting bag and the connector 140. However, it will be appreciated that it may be detachably attached to the collecting bag 130 and/or the connector 140. Additionally, or as a supplement, a downstream hose 138 may be provided between the downstream connector 106 and the dry chamber 100. Corrugations 136 on the outer surface of the connector 140 are adapted to engage the inner surface of the downstream connector. The length of the downstream hose 138 may be in the range of 10-200 cm, such as 50-150 cm such as 100-120 cm, such as 50 cm, as 90 cm such as 120 cm such as 140 cm such as 180 cm.

In some embodiments of the invention according to the first aspect, the length of the upstream hose and the downstream hose are substantially identical e.g. the longer of the two is shorter than 200% of the shorter, such as 150%, such as 125%, such as 110%. If the system is used in connection with non-bedridden patients, the longer of the two may be up to 500% of the shorter of the two, such as 300%.

The collecting bag 130 comprises a drainage passage 142 for emptying the collecting bag 130.

FIG. 4 discloses a use situation wherein a patient 144 is about to be moved from one bed to another. Accordingly, the medical personnel have positioned the collecting bag 130 on an upper surface of the patient, in this case on the legs, whereby orientation of the collecting bag is changed from a substantially upright position to a substantially horizontal position.

In conventional systems the dry chamber forms an integral part of the collecting bag whereby its orientation is also changed from a substantially upright to a substantially horizontal position. Accordingly, fluid contained in the collecting bag and/or the lower part of the dry chamber is brought into contact with the dripping surface. This allows bacteria to migrate from the collecting bag to the body cavity via dripping surface and the draining device. This increases the risk of infections.

In the present invention, the dry chamber 100 and the collecting bag 130 are interconnected by a downstream hose 138. Accordingly, the dry chamber 100 may be maintained in its substantially upright position while the collecting bag 130 is positioned in a substantially horizontal position, as illustrated in FIG. 4. This prevents bacteria from migrating from the collecting bag 130 or the lower part of the dry chamber 100 to the dripping surface 108.

1. A dry chamber for a modular drainage system, the dry chamber comprising:
a substantially rigid housing;
an upstream connector in fluid connection with a dripping surface from which fluid may drip inside the dry chamber when the dry chamber is positioned in a substantially upright position; and
a downstream connector fluidly connected to the dry chamber.

2. A dry chamber according to claim 1, wherein the downstream connector and the dry chamber are interconnected by a flexible annular element defining a conduit and allowing the downstream connector to be elevated to a position above the dry chamber, when the dry chamber is positioned in said substantially upright position.

3. A dry chamber according to claim 2, wherein the flexible annular element interconnecting the dry chamber and the downstream connector forms an integral part of dry chamber.

4. A dry chamber according to claim 1, wherein the flexible annular element interconnecting the dry chamber and the downstream connector is detachably connected to the dry chamber.

5. A dry chamber according to claim 1, wherein the upstream connector forms an integral part of the dry chamber.

6. A dry chamber according to claim 1, wherein the upstream connector and the dry chamber are interconnected by a flexible annular element defining a conduit.

7. A dry chamber according to claim 6, wherein the flexible annular element interconnecting the upstream connector and the dry chamber is detachably connected to the dry chamber.

8. A dry chamber according to claim 1, wherein the upstream connector is adapted to be connected to a corresponding connector of a device for draining body fluids.

9. A dry chamber according to claim 1, wherein the downstream connector is adapted to be connected to a corresponding connector of a collecting bag for collecting body fluids.

10. A dry chamber according to claim 1, wherein the dry chamber comprises an indicator for indicating a chemical or biological property of a fluid contacting the indicator.

11. A dry chamber according to claim 10, wherein the indicator is provided downstream of the dripping surface.

12. A dry chamber according to claim 10, wherein the dry chamber is adapted to allow replacement of the indicator.

13. A dry chamber according to claim 10, wherein the indicator is a pH-indicator.

14. A dry chamber according claim 1, further comprising an antireflux valve for preventing fluid from flowing in an upstream direction of the dry chamber.

15. A kit of elements comprising a dry chamber having an upstream connector and a downstream connector, the kit further comprising at least one of:
a collecting bag for collecting body fluids, comprising a connector connectable to the downstream connector of the dry chamber;
a device for draining body fluids, comprising a connector adapted to be connected to the upstream connector of the dry chamber;
an annular element for fluidly interconnecting the upstream connector and a device for draining fluids; and
an annular element for fluidly interconnecting the downstream connector and a collecting bag.

16. Use of a flexible annular element for fluidly interconnecting a dry chamber and a bag for collecting
